(19) United States
(12) Patent Application Publication
(10) Pub. No.: US 2024/0115263 A1
(43) Pub. Date: Apr. 11, 2024

(54) DEVICE, SYSTEMS, AND METHODS FOR MOVING AND/OR HOLDING ANATOMICAL STRUCTURES IN APPOSITION

(71) Applicant: BOSTON SCIENTIFIC SCIMED, INC., Maple Grove, MN (US)

(72) Inventors: Jeff Gray, Sudbury, MA (US); Katharine Eckerline, Plymouth, MN (US); Kyle P. Moore, Hopkinton, MA (US); Natalie Byrom, Boston, MA (US); Mark Phillip Olson, New Brighton, MN (US); Raymond David Gessler, III, Roberts, WI (US); Nathan Stenger, St. Paul, MN (US); Matthew Maciej, Rogers, MN (US)

(73) Assignee: BOSTON SCIENTIFIC SCIMED, INC., MAPLE GROVE, MN (US)

(21) Appl. No.: 18/377,339

(22) Filed: Oct. 6, 2023

Related U.S. Application Data
(60) Provisional application No. 63/414,129, filed on Oct. 7, 2022.

(51) Int. Cl. A61B 17/11; A61B 17/34

(52) U.S. Cl. CPC A61B 17/11; A61B 17/3478; A61B 2017/0034; A61B 2017/1103

(57) ABSTRACT

An apposition device having a first elongate element movably coupled with a second elongate element. The second elongate element is deployable distal to the structures to be apposed, in a position transverse to the first elongate element. The first elongate element may be pulled to pull the second elongate element to move the structures into apposition. A third element may be positioned proximal to the structures to hold the structures in apposition. The third element may be a ratcheting element fixable in place with respect to the first elongate element. The apposition device may be deployed with a fine gauge needle. The second elongate element may be deployed from a side port in the needle facilitating deployment, and/or movement of the second elongate element with respect to the first elongate element and into a deployment configuration. Such arrangement may allow control of the first elongate element independent of the needle.

### CROSS-REFERENCE TO RELATED APPLICATIONS

This application claims the benefit of priority under 35 U.S.C. § 119 to U.S. Provisional Application No. 63/414,129, filed Oct. 7, 2022, the entire disclosure of which is hereby incorporated by reference herein for all purposes.

### FIELD

The present disclosure relates generally to the field of devices, systems, and methods for moving anatomical structures into apposition with each other and/or holding anatomical structures in apposition with each other. More particularly, the present disclosure relates to medical devices, systems, and methods of delivering and deploying an anchor configured to hold anatomical structures in apposition.

### BACKGROUND

Complex endoscopic ultrasound (EUS) guided procedures (such as in the gastrointestinal tract) require adequate and accurate positioning while maintaining clear ultrasound imaging in order to perform a procedure safely and effectively to the desired access site. Such procedures may include various tasks, including, without limitation, cutting, cauterizing, delivering a treatment device, etc., which typically require adequate and accurate positioning and control. Although endoscopic imaging modalities, such as fluoroscopy and endoscopic ultrasound (EUS), have the ability to view anatomical structures beyond the proximate, visually accessible, tissue in front of the endoscope, therapeutic endoscopic procedures may be limited by the difficulty of controlling tissue beyond the tissue plane visually accessible to the endoscope. Different anatomical sites pose unique challenges, such as with positioning, distance, and shape. For example, placing a stent between two non-adherent anatomical structures (e.g., tissue walls) such as in a gastrojejunostomy, hepaticogastrostomy, or gallbladder drainage into either the stomach or duodenum is technically challenging due to the lack of tools to visualize, stabilize, and, in some cases, inflate the target site. For instance, in certain procedural situations, it is challenging to place a stent in proper alignment with non-adherent anatomical structures (e.g., tissue walls). For instance, advancement of a stent between two non-adherent anatomical structures may push the distal non-adherent anatomical structure away from the proximal non-adherent anatomical structure. In some situations, the stent may not be able to securely hold the non-adherent structures in apposition as desired, or necessary, for the procedure. Moreover, once a site is accessed, physicians need to be able to maintain the access without the proximal or distal structure moving away from or slipping with respect to the proximal structure, which may result in losing the access site. In some instances, the distal site cannot readily be re-accessed if it moves away from the proximal structure, leaving the patient with a perforation in the distal structure and potentially harmful leakage of bodily fluid therefrom. All of these challenges can lead to a failed procedure with serious complications in a patient population already in extremely poor health where there is no room for failure. Improvement to delivery and deployment of devices which move anatomical structures into apposition and/or hold anatomical structures into apposition would be welcome in the field.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a system is provided for moving and holding a proximal anatomical structure and a distal anatomical structure in apposition with respect to each other. The system includes an apposition device comprising a first elongate element and a second elongate element movably coupled with respect to each other; a needle with a lumen therethrough in which at least the second elongate element is positionable; and a pusher within the needle lumen positioned proximal to the second elongate element. In some embodiments, at least one component of the system is structured and configured to facilitate movement of the second elongate element with respect to the first elongate element from a delivery position generally along the first elongate element to a deployed configuration transverse to the first elongate element.

In some embodiments, a side port is defined in a wall of the needle configured for deployment of the second elongate element therethrough. In some embodiments, a ramp is provided within the needle lumen along the side port to facilitate deployment of the second elongate element from the needle lumen and pivoting of the second elongate member into the deployed configuration. In some embodiments, the ramp is formed from a portion of the needle wall pushed inwardly into the needle lumen to form the port. In some embodiments, the side port is defined proximally to a distal end of the needle a distance allowing deployment of the second elongate element within a small space distal to the distal anatomical structure.

In some embodiments, the apposition device further comprises a third element positioned proximal to the second elongate element and distal to the pusher and configured to push the second elongate element into the deployed position when pushed distally by the pusher.

In some embodiments, a third element configured and positioned to hold the anatomical structures in apposition. In some embodiments, the third element is an expanded element associated with the first elongate element and positioned proximal to the proximal anatomical structure to hold the anatomical structures in apposition. In some embodiments, the first elongate element and the third element are configured to be fixed with respect to each other to hold the structures in apposition between the third element and the third element. In some embodiments, the first elongate element and the third element include interengaging ratchet features.

In some embodiments, the needle is a fine gauge needle.

In accordance with various principles of the present disclosure, a system is provided for moving and holding a proximal anatomical structure and a distal anatomical structure in apposition with respect to each other. The system includes an apposition device comprising a first elongate element and a second elongate element movably coupled with respect to each other; and a needle with a lumen therethrough in which at least the second elongate element is positionable; wherein a side port is defined in a wall of the needle configured for deployment of the second elongate element therethrough.

In some embodiments, a ramp is provided within the needle lumen along the side port to facilitate deployment of the second elongate element from the needle lumen and pivoting of the second elongate member into the deployed configuration. In some embodiments, the ramp is formed from a portion of the needle wall pushed inwardly into the needle lumen to form the port.

In some embodiments, the system includes a third element configured and positioned to hold the anatomical structures in apposition, the third element comprising an expanded element fixable with respect to the first elongate element proximal to the proximal anatomical structure to hold the anatomical structures in apposition.

In accordance with various principles of the present disclosure, a method of holding a first anatomical structure and a second anatomical structure in apposition with respect to each other uses an apposition device comprising a first elongate element and a second elongate element movably coupled with each other. In some aspects, the method includes delivering the second elongate element of the apposition device within a lumen of a needle to a deployment position distal to the second anatomical structure; extending the second elongate element out a side port formed in a wall of the needle; causing the second elongate member to move to a position transverse to the first elongate element; proximally withdrawing the needle proximal to the anatomical structures; and pulling the first elongate element proximally to draw the second elongate element proximally to move the second anatomical structure into apposition with the first anatomical structure.

In some aspects, the first elongate element extends proximally along the outside of the needle, the method further including pulling the first elongate element proximally to cause the second elongate member to pull the second anatomical structure in apposition with the first anatomical structure. In some aspects, the method further includes positioning a third element with respect to the first elongate element and the first anatomical structure to hold the anatomical structures in apposition.

In some aspects, the method further includes advancing a pusher distally through the needle lumen to push the second elongate element out of the side port. In some aspects, the method further includes pushing the second elongate element along a ramp within the needle lumen to advance the second elongate element out the side port defined in the needle wall.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers with a ′ after one of the reference numbers, and with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an embodiment of an apposition device formed in accordance with various aspects of the present disclosure and positioned in a schematic representation of a deployment environment.

FIG. 2 illustrates an elevational view of an example of an embodiment of an apposition device formed in accordance with various principles of the present disclosure.

FIG. 3 illustrates a perspective view of a delivery needle which may be used to deliver an apposition device such as illustrated in FIG. 2.

FIG. 4A and FIG. 4B illustrate examples of manners of loading an apposition device such as illustrated in FIG. 2 into a delivery needle such as illustrated in FIG. 3.

FIG. 5A, FIG. 5B, and FIG. 5C illustrate sequential stages of delivery and deployment of an apposition device such as illustrated in FIG. 2.

FIG. 6 illustrates a perspective view of an example of an embodiment of an apposition device formed in accordance with various principles of the present disclosure with an associated delivery needle.

FIG. 7 illustrates an apposition device such as illustrated in FIG. 6 being deployed in a schematic representation of a deployment environment.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, etc.) such location or site.

In accordance with various principles of the present disclosure, non-adherent anatomical structures (such as tissue walls of the same or different anatomical structures such as organs, vessels, muscles, etc.) are held in apposition with an apposition device. The medical professionals are thereby enabled to anchor a site safely while maintaining endoscopic ultrasound imaging, and to establish and maintain access to the non-adherent anatomical structures during device exchanges to enhance the safety profile of the procedure (e.g., an EUS guided procedure). It will be appreciated that reference is made to non-adherent, although the present disclosure need not be so limited and may apply, as well, to anatomical structures which may adhere to each other, at least to some extent.

In accordance with various principles of the present disclosure, the apposition device includes a first elongate element and a second elongate element. The second elongate element and a portion of the first elongate element are passed from a position proximal to the proximal of two adjacent non-adherent anatomical structures, through the non-adherent anatomical structures, and to a position distal to the two non-adherent anatomical structures. The pathway/passage through the non-adherent anatomical structures along which the second elongate element passes to reach its position distal to the distal of the non-adherent anatomical structures is referenced herein as a “puncture site” for the sake of convenience and without intent to limit. The second elongate element is delivered to the position distal to the non-adherent anatomical structures in a position substantially parallel to the longitudinal axis of the first elongate element and/or the needle (to facilitate delivery and/or passage through the non-adherent anatomical structures). It will be appreciated that reference to a longitudinal axis herein may be to the longitudinal axis of the needle, the first elongate element, and/or the system, as may be appreciated by of ordinary skill in the art in view of the context of the reference. Once the second elongate element is distal to the distal of the non-adherent anatomical structures, the second elongate element is pivoted with respect to the first elongate element (e.g., about an axis transverse to the longitudinal axis of the first elongate element) into a position transverse to the longitudinal axis of the first elongate element. The second elongate element may thus be considered a T-bar or T anchor element. It will be appreciated that the second elongate element may be additionally or alternatively rotationally coupled to the first elongate element (e.g., about the longitudinal axis of the first elongate element).

The second elongate element is formed of a material resistant to being pulled through the puncture site. In some embodiments, the second elongate element is more rigid than the first elongate element. In some embodiments, the first elongate element is a flexible elongate element, such as a suture, tether, wire, string, cord, thread, band, etc., such terms being used interchangeably herein, reference typically made herein to a suture for the sake of convenience and without intent to limit. In some embodiments, the second elongate element is a bar formed of a biocompatible metal (e.g., nitinol, stainless steel, surgical alloys, etc.) or a biocompatible polymer (e.g., Polycarbonate, ABS, Rigid PVC, Acetal, Rigid Urethane, polyether ether ketone (PEEK), Nylon, Polyimide, etc.) or combinations thereof.

In accordance with various principles of the present disclosure, the apposition device is deliverable with a needle providing a length, presence of stylet, cutting tip, echogenicity, etc., suitable for use in bringing tissue into apposition, such as a needle used in fine needle aspiration procedures (an “FNA” needle) or a needle used in fine needle biopsies (an “FNB” needle). An FNA needle is typically manufactured in a configuration which is compatible with endoscopic procedures (e.g., compatible for use with an echo duodenoscope). Typically, an FNA needle is a minimally invasive, generally flexible needle with a lumen extending therethrough and through which a stylet may extend. The FNA needle may be a 27 gauge needle (typically approximately with a 0.413 mm outer diameter, 0.21 mm inner diameter, and 0.102 mm wall thickness), but may be a 25 gauge needle (typically approximately with a 0.515 mm outer diameter, 0.153 mm inner diameter, and 0.127 mm wall thickness), a 22 gauge needle (typically approximately with a 0.718 mm outer diameter, 0.413 mm inner diameter, and 0.152 mm wall thickness), or even a 19 gauge needle (typically approximately with a 1.067 mm outer diameter, 0.686 mm inner diameter, and 0.191 mm wall thickness), or other size acceptable for delivery of an apposition device in accordance with various principles of the present disclosure as may be appreciated by those of ordinary skill in the art.

In some aspects, the apposition device is delivered with an FNA needle having a side port in communication with a lumen extending longitudinally through the needle. The second elongate element of the apposition device is insertable through the side port into the needle lumen to be delivered to the deployment site distal to the distal non-adherent anatomical structure. The first elongate element may extend out the side port of the needle, and proximally towards the proximal end of the needle for access and control by a medical professional. It will be appreciated that terms such as control, manipulate, move, navigate, etc., and other grammatical forms thereof, may be used interchangeably herein without intent to limit. Once the distal end of the needle is positioned at an appropriate deployment position, the first elongate element may be pulled to withdraw and to deploy the second elongate member. The second elongate element may then pivot into a position transverse to the first elongate element, such as along a distal surface of the distal non-adherent anatomical structure and transverse to the passage through the non-adherent anatomical structures through which the second elongate element had been delivered.

In some aspects, an apposition system includes an apposition device and a deployment system configured to facilitate deployment of the apposition device. In some embodiments, the apposition device is configured to maintain non-adherent anatomical structures in apposition with each other with the use of a third element of the apposition device. The third element may be a locking or fixation element configured to hold the non-adherent anatomical structures in place with respect to each other, such as by being fixed with respect to the second elongate element. The third element remains proximal to the proximal of the non-adherent anatomical structures. In some embodiments, the third element is movable with respect to the first and second elongate elements. The first element may be pulled proximally and/or the third element may be advanced distally in a direction toward the second element, such as with a deployment lumen, to cinch the apposition device to hold the non-adherent anatomical structures in apposition. In some embodiments, the third element is configured to be locked in place with respect to the first element. For instance, the third element and the first element may have cooperating engaging elements holding the third element in a selected position with respect to the first element holding the non-adherent anatomical structures in apposition.

Various embodiments of devices, systems, and methods for bringing non-adherent anatomical structures into apposition and/or holding non-adherent anatomical structures in apposition will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. The following description is of illustrative examples of embodiments only, and is not intended as limiting the broader aspects of the present disclosure.

Turning now to the drawings, an example of an embodiment of an apposition device 100 is illustrated in a schematic representation of an example of a deployment site holding, in apposition, non-adherent anatomical structures PS, DS of a patient's body. In particular, the illustrated example of an embodiment of an apposition device 100 is depicted as deployed across a proximal anatomical structure PS and a distal anatomical structure DS to hold such structures together in apposition. In the illustrated example of an embodiment, the proximal anatomical structure PS is the stomach S (e.g., a wall thereof), and the distal anatomical structure DS is the jejunum J (e.g., a wall thereof) of a patient. However, it will be appreciated that principles of the present disclosure are applicable to other anatomical structures, such as, without limitation, non-adherent anatomical structures to be held in apposition. Typically, one such non-adherent anatomical structure may be considered a proximal non-adherent anatomical structure and the other non-adherent anatomical structure may be considered a distal non-adherent anatomical structure, such directions being generally relative to the medical professional bring such structures into apposition and/or the direction from which the apposition device 100 approaches such structures to be deployed with respect to such structures. The non-adherent anatomical structures PS, DS are illustrated as tissue walls, though need not be so limited. Once the apposition device 100 has been deployed, further procedures, such as creation of an anastomosis between the stomach S and the jejunum J, and/or drainage of materials from one non-adherent anatomical structure to the other non-adherent anatomical structure, may be performed with the apposition device 100 maintaining the structures in apposition with respect to each other.

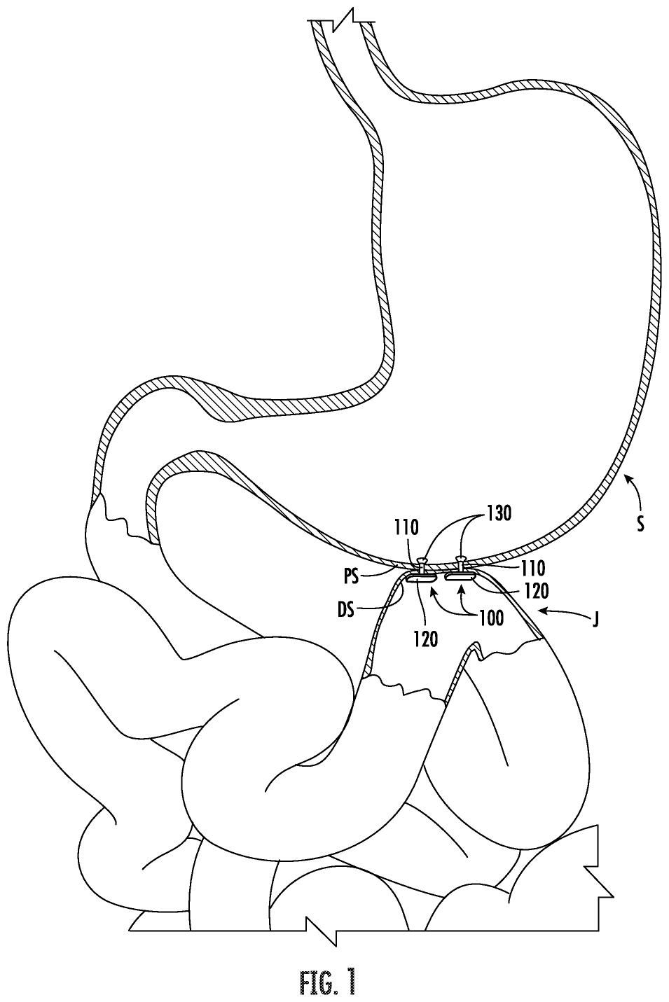

The example of an embodiment of an apposition device 100 illustrated in FIG. 1 has a first elongate element 110 extending through the proximal non-adherent anatomical structure PS and the distal non-adherent anatomical structure DS, a second elongate element 120 distal to the distal non-adherent anatomical structure DS, and a third element 130 along a portion of the first elongate element 110 proximal to the proximal non-adherent anatomical structure PS. More particularly, the second elongate element 120 is positioned and configured (sized, shaped, dimensioned, formed of a material, etc.) to resist being pulled proximally through the puncture site. For instance, the second elongate element 120 may be positioned transverse to the first elongate element 110 and sufficiently long, resistant to flexing, etc., to resist being pulled proximally through the puncture site. The third element 130 is positioned and configured (sized, shaped, dimensioned, formed of a material, etc.) to resist being pulled distally through the puncture site, and to hold the apposition device 100 in place with respect to the non-adherent anatomical structures PS, DS. For instance, the third element 130 may be an enlarged element positioned against a proximal side of the proximal non-adherent anatomical structure PS at a position with respect to the second elongate element 120 to hold the non-adherent anatomical structures PS, DS in apposition with the appropriate holding force. The third element 130 may be a separate element movable (e.g., slidable) with respect to the first elongate element 110 to be selectively positioned with respect to the second elongate element 120 to achieve the desired retention of the non-adherent anatomical structures PS, DS in apposition with respect to each other. Alternatively, the third element 130 may simply be a knot made in the first elongate element 110 or other formation created in situ (e.g., weld, crimp, application of material, etc.) to modify the first elongate element 110 to hold the non-adherent anatomical structures PS, DS in apposition. The second elongate element 120 and the third element 130 may be considered to function in conjunction with each other to hold the non-adherent anatomical structures PS, DS in apposition.

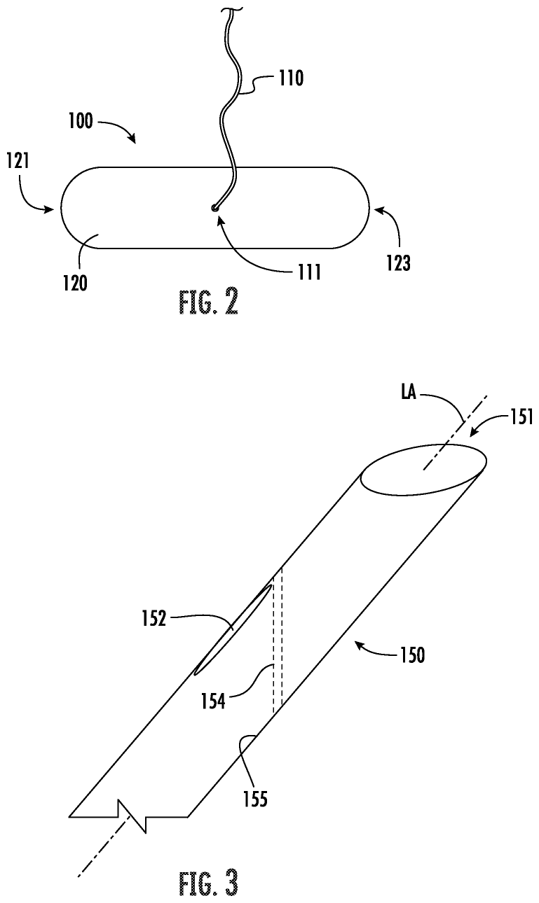

In the example of an embodiment of an apposition device 100 illustrated in FIG. 2, the first elongate element 110 is a flexible elongate element, such as a suture, tether, wire, string, cord, thread, band, etc., capable of extending through the non-adherent anatomical structures to be held in apposition without damaging or otherwise interfering with the functioning of such structure. The first elongate element 110 may have a flexibility selected to not impede advancement of the device (e.g., needle) delivering the first elongate element 110 and/or not to impede advancement of the first elongate element 110 through anatomical tissue. The first elongate element 110 may have a length enabling a distal end 111 thereof to be deployed at the non-adherent anatomical structures, and a proximal end extendable (directly or via another control element) to a control handle (formed in a manner known to those of ordinary skill in the art), or outside the patient's body for manual control (e.g., grasping of the first elongate element 110 in the hand of a medical professional directly or via another element). The flexibility of the first elongate element 110 allows the first elongate element 110 to fit within the lumen of a delivery device or along a delivery device navigating transluminally through the patient's body.

In the example of an embodiment of an apposition device 100 illustrated in FIG. 2, the second elongate element 120 is an elongated element pivotably coupled to the first elongate element 110. The distal end 111 of the first elongate element 110 may be coupled with the second elongate element 120 in any of a variety of manners known to those of ordinary skill in the art, such as bonding (e.g., with an adhesive, or ultrasonic bonding, or welding, or another chemical-type bond) and/or a mechanical coupling (e.g., crimping; extending through an aperture through the second elongate element 120 and having an enlarged end, such as a knot or enlarged adhesive bead, preventing withdrawal therefrom; or another type of interference fit), the present disclosure not being limited in this manner. The second elongate element 120 may be coupled with the first elongate element 110 to be movable between a delivery position generally aligned or otherwise extending along the first elongate element 110 to facilitate delivery (e.g., transluminally) to a deployment site, such as to facilitate delivery through the puncture site, and a deployed position generally transverse to the puncture site. In the deployed position, the second elongate element 120 may be anchored with respect to the distal of the non-adherent anatomical structures (e.g., against a distal surface of the distal of the non-adherent anatomical structures). For instance, in the deployed position, the second elongate element 120 may extend (e.g., along its elongated extent/longer dimension) across the deployment site and transverse to the puncture site. In some embodiments, the first elongate element 110 is coupled to the second elongate element 120 along a midregion of the second elongate element 120 (between the ends 121, 123 of the second elongate element 120). The position at which the first elongate element 110 is coupled with the second elongate element 120 may be midway between the ends 121, 123 of the second elongate element 120, or any other location facilitating pivoting of the second elongate element 120 upon proximal pulling on the first elongate element 110 to pull the second elongate element 120 against an anatomical structure. The second elongate element 120 preferably is resistant to backing out of the deployment site, such as by being proximally drawn through the puncture site, after deployment distal to the non-adherent anatomical structures. For instance, the second elongate element 120 may be sufficiently resistant to flexing so that the second elongate element 120 cannot be pulled through the apposition device passage. Optionally, the second elongate element 120 is less flexible than the first elongate element 110.

The illustrated example of an embodiment of a second elongate element 120 is in the form of a bar or slug or anchor or tubular element or other generally elongated element capable of moving (e.g., pivoting) with respect to the first elongate element 110 and anchoring against a distal side of the distal of the non-adherent anatomical structures to be held in apposition by the apposition device 100. The second elongate element 120 is sized, shaped, configured, and/or dimensioned to anchor the apposition device 100 with respect to the distal of the apposed non-adherent anatomical structures. In some embodiments, the second elongate element 120 is a solid bar (flat, cylindrical, or otherwise). Alternatively, the second elongate element 120 may be tubular with a lumen therethrough. The second elongate element 120 may be long enough to facilitate anchoring with respect to an anatomical structure (e.g., tissue wall) and remain in place in tissue, yet short enough to readily exit the device with which the second elongate element 120 is delivered (e.g., approximately 1-2 cm in length). The second elongate element 120 may be formed of any preferably biocompatible material such as a metal (e.g., Nitinol or stainless steel), polymer, or combination thereof. In some embodiments, the material of the second elongate element 120 is selected to be visible under ultrasound and/or fluoroscopy to facilitate placement, such as to confirm orientation of the second elongate element 120 in a desired anchoring position with respect to the non-adherent anatomical structures. As may be appreciated by those of ordinary skill in the art, the texture and/or rigidity and/or other properties of the second elongate element 120 may be selected to improve visibility under ultrasound or with other imaging techniques. For instance, the second elongate element 120 may have surface treatment configured to optimize an ultrasound response for orientation, placement, etc., of the second elongate element 120. For instance, an imaging technique may be used to confirm the desired distance between the apposed non-adherent anatomical structures, such as by viewing the delivery device (e.g., an endoscope) proximal to the non-adherent anatomical structures, and the second elongate element 120 distal to the apposed non-adherent anatomical structures. The first elongate element 110 may be pulled proximally to feel resistance of the second elongate element 120 with respect to the non-adherent anatomical structures to confirm anchoring of the second elongate element 120.

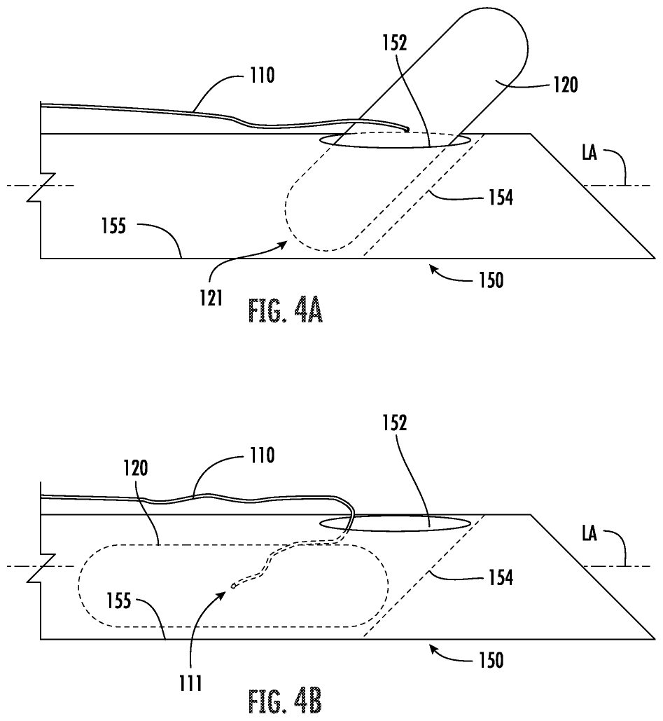

In accordance with various principles of the present disclosure, an apposition device 100 such as illustrated in FIG. 2 may be delivered with a needle. The needle may be a fine gauge needle, such as used for fine needle aspirations as known to those of ordinary skill in the art. In accordance with various further principles of the present disclosure, a modified needle 150, such as illustrated in FIG. 3, may be used. The modified needle 150 has a side port 152 defined in a wall thereof allowing transverse access to the lumen 155 defined longitudinally therein (along the longitudinal axis LA of the needle 150). The side port 152 may be formed in any of a variety of manners known to those of ordinary skill in the art, such as punching, laser cutting, etc. In some embodiments, a portion of a wall of the needle 150 is cut and pressed inwardly to form the side port 152 as well as a ramp 154 extending into the lumen 155 of the needle 150. The ramp 154 may be dimensioned and positioned within the needle lumen 155 so as to maintain functionality of the lumen 155, such as by not occluding the lumen 155. For instance, the ramp 154 may be dimensioned/positioned so as not to block passage of materials (e.g., gases, liquids, or other media) from a proximal end of the needle 150 (which may be connected to a fluid source and/or a vacuum source) to the distal end 151 of the needle 150. The side port 152 allows delivery of the second elongate element 120 within the lumen 155 of the needle 150 with the first elongate element 110 extending outside and proximally along the needle 150. Such configuration allows an apposition system (including the apposition device 100 and various delivery and/or deployment devices configured to deliver and deploy the apposition device 100, including, without limitation, the needle 150 and other devices described below) to maintain a sufficiently narrow profile to be delivered transluminally within a body to the selected deployment site for the apposition device 100, as may be appreciated by those of ordinary skill in the art. Optionally, the ramp 154 may be dimensioned and positioned to facilitate movement of the second elongate element 120 into and out of the lumen 155 as described in further detail below.

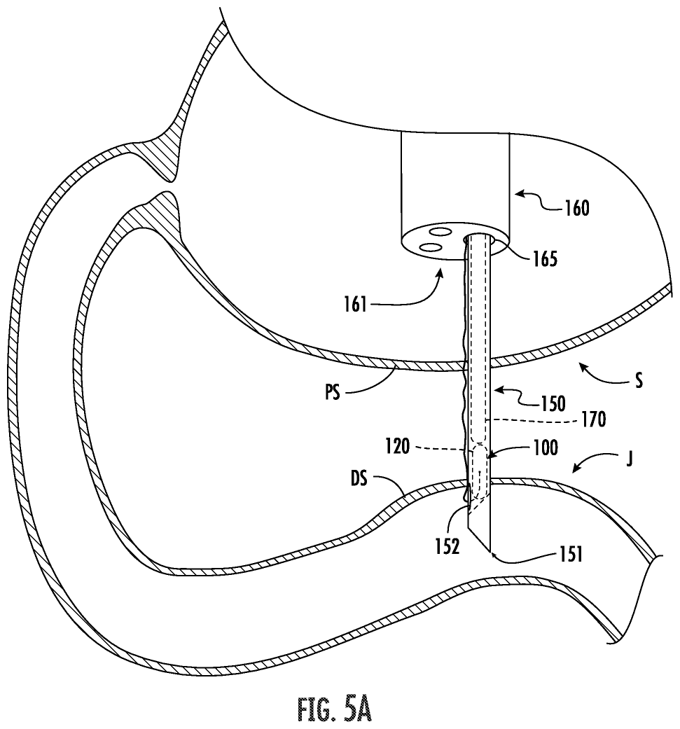

An apposition device 100 (such as the apposition device 100 illustrated in FIG. 2) may be delivered to a deployment site with a needle 150 with a side port 152 (such as illustrated in FIG. 3) by back-loading the second elongate element 120 into the lumen 155 of the needle 150 via the side port 152 in a manner such as illustrated in FIG. 4A and FIG. 4B. As illustrated in FIG. 4A, an end 121 of the second elongate element 120 is inserted into the side port 152 of the needle 150 with the second elongate element 120 (e.g., the longitudinal extent of the second elongate element 120 extending between the ends 121, 123) transverse to the longitudinal axis LA of the needle 150. The second elongate element 120 may be guided into the needle lumen 155 by a ramp 154 within the lumen 155. The ramp 154 may be formed or provided within the needle lumen 155 to facilitate loading of the second elongate element 120 therein as well as deployment therefrom when the needle 150 reaches the deployment site. The second elongate element 120 may be positioned within the lumen 155 for delivery substantially aligned with the longitudinal axis LA, such as illustrated in FIG. 4B. The first elongate element 110 may be extended from a position within the lumen 155 (the distal end 111 of the first elongate element 110 being coupled with the second elongate element 120 within the needle lumen 155) out through the side port 152. The first elongate element 110 may then be extended proximally along (e.g., alongside) the needle 150 to a location from which the proximal end of the first elongate element 110 may be accessed to control the apposition device 100. One of ordinary skill in the art will appreciate that selection of appropriate geometries of the second elongate element 120 and the side port 152 and the ramp 154 as well as the diameter/thickness of the first elongate element 110 facilitate the desired interaction of the components of the apposition system in delivering and deploying the apposition device 100.

Arrangement of the first elongate element 110 outside the lumen 155 of the needle 150 may allow the first elongate element 110 to be controlled substantially independently of the needle 150. For instance, it may be desirable to adjust the position of the apposition device 100 by pulling the first elongate element 110 to pull the second elongate element 120 to move the non-adherent anatomical structures into apposition with respect to each other. Positioning of the first elongate element 110 outside the needle 150 may facilitate control of the first elongate element 110 and/or reduce potential interference by the needle 150. Additionally or alternatively, running the first elongate element 110 proximally from the second elongate element 120 outside the needle 150 allows withdrawal of the needle 150 independently of the first elongate element 110 such as once the second elongate element 120 has been deployed, and/or allows adjustment of the first elongate element 110 and the second elongate element 120 independent of the needle 150 once deployed. Because the first elongate element 110 does not extend through the needle 150, the needle 150 may be proximally withdrawn from the non-adherent anatomical structures without interfering with the apposition device 100. If desired, the needle 150 may be withdrawn, another apposition device may be loaded with respect to the needle 150, and the needle 150 advanced to the non-adherent anatomical structures held in place by the first-delivered apposition device 100 to deliver another apposition device (e.g., adjacent and spaced apart from the first-delivered apposition device 100) to provide additional securement of the non-adherent anatomical structures PS, DS, such as illustrated in FIG. 1.

Positioning of at least a portion of an apposition device 100 through a side port 152 of a needle 150 allows deployment of such portion of the apposition device 100 from the side port 152 rather distally than through the distal end 151 of the needle 150. Such arrangement may protect the first elongate element 110 and the second elongate element 120 from a potentially sharp needle distal end 151. Moreover, by deploying the second elongate element 120 sideways with respect to the longitudinal axis LA of the needle 150, less space may be needed distal to the distal end 151 of the needle 150 at the deployment site. For instance, if the distal of the non-adherent anatomical structures to be brought into apposition is a jejunum J or a vessel or small organ or otherwise, the deployment site for the second elongate element 120 does not afford a lot of room for the distal end 151 of the needle 150 and deployment of the apposition device 100 distally therefrom, Moreover, the side port 152 may be formed near or adjacent a distal end 151 of the needle 150 to facilitate delivery of the second elongate element 120 within small spaces so that the distal end 151 of the needle 150 need not be advanced too far to position the side port 152 distal to the non-adherent anatomical structures for deployment of the second elongate element 120.

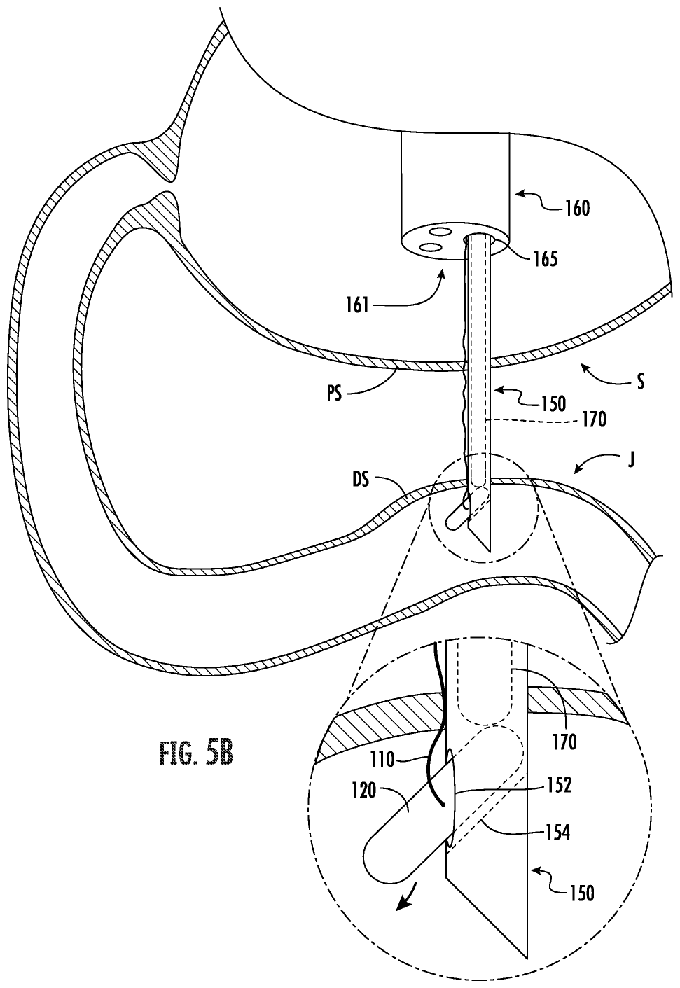

An example of a manner in which an example of an embodiment of an apposition system formed in accordance with various principles of the present disclosure may be used to deliver and/or deploy an example of an embodiment of an apposition device 100 (which may, in some aspects, be considered a part of the apposition system) is illustrated in FIGS. 5A-5C.

The apposition device 100 may be backloaded with respect to a needle 150, such as in a manner as described above (e.g., the second elongate element 120 inserted/loaded into a side port 152 in the needle 150, and the first elongate element 110 extended proximally). The needle 150 and the apposition device 100 may then be introduced into an endoscope 160 (into a working channel 165 of the endoscope 160). The endoscope 160 is advanced to the desired/indicated/selected deployment site in a manner known to those of ordinary skill in the art. For instance, natural orifice transluminal endoscopic surgery (NOTES) may be used to extend the endoscope 160 into a patient's stomach S, as illustrated in FIG. 5A, via the patient's mouth (not shown). The deployment site for the apposition device 100 may be determined in any of a variety of manners known to those of ordinary skill in the art. For instance, ultrasound, fluoroscopy, or other imaging techniques may be used. In some instances, a beacon (e.g., a light beacon) may be positioned at the deployment site for the second elongate element 120 (distal to the distal of the non-adherent anatomical structures PS, DS to be brought into apposition with the apposition device 100).

Once the distal end 161 the endoscope 160 is positioned for deployment of the apposition device 100 to bring non-adherent anatomical structures PS, DS into apposition, the needle 150 (preloaded with an apposition device 100) may be advanced distally out of the working channel 162 of the endoscope 160. The needle is extended through the proximal anatomical structure PS and through the distal anatomical structure DS to deploy the apposition device 100 distal to the distal of the non-adherent anatomical structures PS, DS. For example, in the example illustrated in FIG. 5A, the needle 150 is extended through the wall of the stomach S and into the jejunum J to deploy the apposition device 100 within the jejunum J. When using a needle 150 such as illustrated in FIG. 3, the distal end 151 of the needle 150 is advanced a sufficient distance so that the side port 152 is distal to the distal anatomical structure DS and spaced from the puncture site therethrough to allow room for deployment of the apposition device 100.

In some embodiments, the location of the needle 150 distal to the distal anatomical structure DS is confirmed before deployment of the apposition device 100. For instance, a portion of the needle 150 and/or the apposition device 100 may include a visualization material or marker such as a radiopaque marker, or may be formed of a material facilitating imaging thereof. Additionally or alternatively, a contrast medium may be delivered and/or a sample withdrawn from the anatomical site of the distal end 151 of the needle 150. Accordingly, as noted above, the ramp 154 within the needle lumen 155 may be sized, shaped, configured, and/or dimensioned to allow passage of media therethrough such as to deliver a contrast medium or otherwise, and/or to aspirate a sample (e.g., bile, to confirm the distal end 151 is within a gall bladder to be treated),

With the distal end 151 of the needle 150 properly positioned, the apposition device 100 may be deployed. For instance, the second elongate element 120 may be deployed out of the side port 152 of the needle 150, such as by pulling the first elongate element 110. Additionally or alternatively, the second elongate element 120 may be deployed by pushing the second elongate element 120 with a pusher 170 (e.g., an elongate element such as a stylet) which extends through the needle lumen 155 proximal to the second elongate element 120 when loaded therein, such as illustrated in FIG. 5B. The pusher 170 may be configured to mate with the second elongate element 120 to facilitate engagement therewith. For instance, the pusher 170 may have a widened blunt end configured to facilitate engagement with and pushing of the second elongate element 120. It will be appreciated that if functionality of the needle lumen 155 is to be maintained, such as described above with reference to the configuration of ramp 154, the pusher 170 is dimensioned to allow passage of materials through the lumen 155 as well. Sliding of the second elongate element 120 along the ramp 154 may facilitate deployment thereof out the side port 152.

Once the second elongate element 120 has been deployed out of the needle 150, the first elongate element 110 may be used to orient the second elongate element 120 with respect to the distal anatomical structure DS to seat against a distal surface thereof. The first elongate element 110 may be coupled to the second elongate element 120 to facilitate orienting of the second elongate element 120. For example, the first elongate element 110 may be coupled between the ends 121, 123 of the second elongate element 120, such as midway therebetween, so that proximal pulling on the first elongate element 110 will orient the second elongate element 120 across the puncture site. The first elongate element 110 may be pulled proximally to draw the distal anatomical structure DS into apposition with the proximal anatomical structure PS, such as illustrated in FIG. 5C. A third element 130 may be positioned proximal to the non-adherent anatomical structures PS, DS to hold the apposition device 100 in position with the structures PS, DS, such as illustrated in FIG. 1. The needle 150 and the optional pusher 170 may be retracted out of the patient's body once the apposition device 100 has been deployed and/or the non-adherent anatomical structures PS, DS are securely held in apposition.

One or more additional apposition devices may be delivered and deployed (e.g., in a manner as described above or otherwise) as desired/medically indicated. For instance, two or more apposition devices may be recommended to achieve a secure hold on certain non-adherent anatomical structures to maintain the structures in apposition. The apposition devices may be spaced apart from one another if desired. The additional apposition devices may be the same as the above-described apposition devices 100 or in different configurations. In some embodiments, the needle 150 may accommodate more than one apposition device 100 so that the needle 150 need not be withdrawn in order to deploy a second, third, etc., apposition device 100 after deployment of the first apposition device 100.

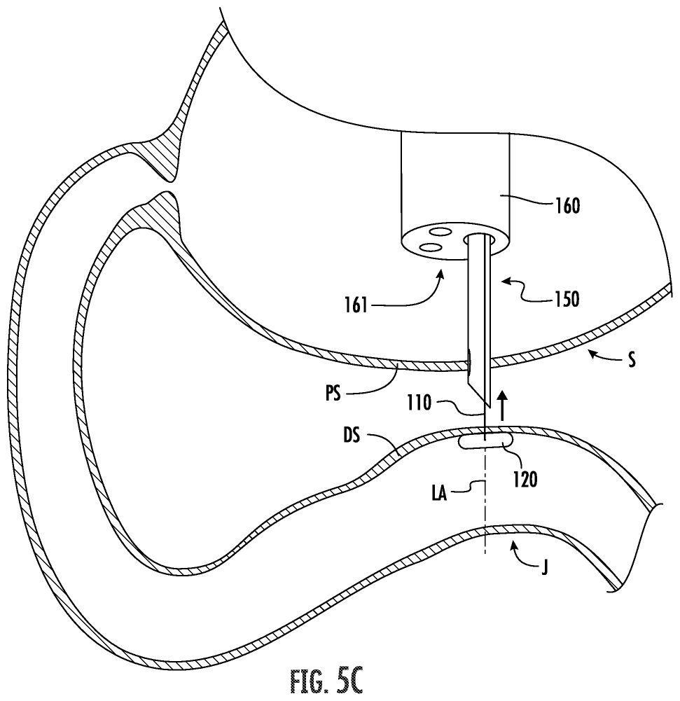

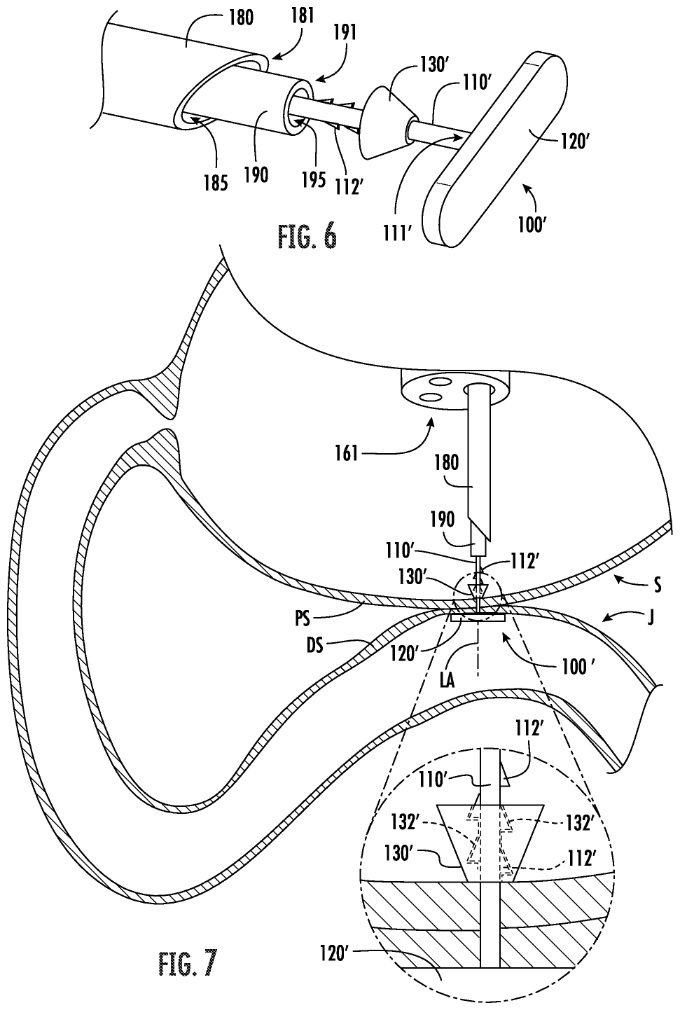

An alternative example of an embodiment of an apposition device 100′ is illustrated in FIG. 6. Like the above-described apposition device 100, the example of an embodiment of an apposition device 100 illustrated in FIG. 6 has a first elongate element 110′ coupled at a distal end 111′ thereof to a second elongate element 120′ and extending proximally, such as for control by a medical professional. The second elongate element 120′ is movably (e.g., pivotably and/or rotationally) coupled with the first elongate element 110′. The second elongate element 120′ is configured to be positioned at a deployment site distal to the distal of non-adherent anatomical structures to be held in apposition. Once at the deployment site, the second elongate element 120′ is movable from a delivery position, generally extending along the first elongate element 110′ (e.g., parallel to the longitudinal axis thereof), to a deployment position generally transverse to the first elongate element 110′, such as illustrated in FIG. 7. In its deployment position, the second elongate element 120′ is positioned relative to at least the distal of the non-adherent anatomical structures to resist inadvertent proximal withdrawal from its deployment site (e.g., to prevent withdrawal of the second elongate element 120′ through a puncture site through which it has been delivered to its deployment site). Materials and other properties of the first elongate element 110′ and second elongate element 120′ may be similar to those described above with respect to the first elongate element 110 and the second elongate element 120 and reference is made thereto for the sake of brevity. In some embodiments, at least the second elongate element 120′ may be molded and/or overmolded with elastic. For instance, an elastic material such as Pebax® may be molded over the second elongate element 120′, such as to allow for some movement of the second elongate element 120′. Alternatively, the second elongate element 120′ may be molded with a more rigid biocompatible polymer such as PEEK. However, in some embodiments, the first elongate element 110′ and the second elongate element 120′ have various other properties such as which may be amenable or suitable for the unique characteristics of the apposition device 100′ in general, such as will now be described.

In accordance with various principles of the present disclosure, the example of an embodiment of an apposition device 100′ illustrated in FIG. 6 includes a third element 130′ movably coupled to/mounted on the first elongate element 110′. Similar to the third element 130 described above, the third element 130′ is configured to hold the apposition device 100′ in a desired position with respect to the non-adherent anatomical structures PS, DS, such as to maintain a desired holding force on the non-adherent anatomical structures to hold them in apposition. In the example of an embodiment illustrated in FIG. 6, the first elongate element 110′ and the third element 130′ are configured to engage, interengage, lock, interlock, mate, fit (e.g., with an interference fit), interfit, etc. (such terms and other grammatical forms thereof being used interchangeably herein without intent to limit) to fix the relative positions thereof. For instance, in the illustrated example of an embodiment, the first elongate element 110′ and the third element 130′ include mating features 112′, 132′ configured to mate and hold the third element 130′ in place with respect to the first elongate element 110. The mating features 112′, 132′ may be sized, shaped, configured, and/or dimensioned to facilitate incremental relative movement of the first elongate element 110′ and the third element 130′, such as to allow incremental adjustment of holding force on the non-adherent anatomical structures by the apposition device 100′. More particularly, the illustrated example of an embodiment of a third element 130′ is in the form of a ratchet washer 130′ configured to engage and ride over external features 112′ on the first elongate element 110. For instance, the mating features 112′, 132′ may be configured (e.g., with asymmetrical ramps such as typical in ratcheting devices) so that once the first elongate element 110′ with the second elongate element 120′ are pulled proximally with respect to the third element 130′, the third element 130′ stays in place and does not slide proximally with respect to the first elongate element 110′ and the second elongate element 120′. The non-adherent anatomical structures PS, DS are thereby between the third element 130′ and the second elongate element 120′ in secure apposition. In the example of an embodiment illustrated in the detail view in FIG. 7, the ratchet washer 130′ has internal features 132′ (bumps, protrusions, etc.) which ride over the external features 112′ on the first elongate element 110 in a direction allowing tightening of the ratchet washer 130′ with respect to the second elongate element 120′ (being drawn closer to the second elongate element 120′), but not allowing reverse movement (loosening or moving away from the second elongate element 120′). Other configurations, such as notches, grooves, molded features, etc., are within the scope and spirit of the present disclosure, particular details of the mating features 112′, 132′ not being critical to general principles of the present disclosure. Various sizes, shapes, configurations, and/or dimensions of the mating features 112′, 132′ may be clinically determined, such as based on any of the various properties of the non-adherent anatomical structures to be held in apposition by the apposition device 100′.

It will be appreciated that the example of an embodiment of an apposition device 100′ illustrated in FIG. 6 may be delivered with/as a system and/or in a manner similar to that described above with reference to FIGS. 5A-5C. For instance, the apposition device 100′ may be delivered to a deployment site with a deployment needle similar to the above-described needle 150. However, in some embodiments, the second elongate element 120′ of the apposition device 100′ is not delivered through a side port of a deployment needle, and, therefore, the deployment needle used with the apposition system with which the apposition device 100′ is delivered and deployed need not include a side port. In contrast with the above-described needle 150, a deployment needle 180 used to deploy the apposition device 100′ illustrated in FIG. 6 may have a needle lumen 185 therein allowing passage of the apposition device 100′ as well as a pusher 190 therethrough and out the distal end 181 of the deployment needle 180. The lumen 185 may be considered to be substantially fully-unobstructed. More particularly, in contrast with the above-described apposition system and method, the apposition device 100′ is delivered with a pusher 190 sized, shaped, configured, and/or dimensioned to fit within and slidably extend through the lumen 185 of the deployment needle 180 to push at least the second elongate element 120′ out the distal end 181 of the deployment needle 180. In the illustrated example of an embodiment, the second elongate element 120′ of the apposition device 100′ is positioned distal to the distal end 191 of the pusher 190. However, other configurations and arrangements are within the scope and spirit of the present disclosure. For instance, the pusher 190 may be in the form of a deployment lumen with the second elongate element 120′ extending partially within a lumen within the pusher 190.

To deploy an apposition device 100′ such as illustrated in FIG. 6, once the distal end 181 of the deployment needle 180 has been advanced distal to the distal of the non-adherent anatomical structures PS, DS to be held in apposition by the apposition device 100′, the second elongate element 120′ is deployed. For instance, the deployment needle 180 and/or the pusher 190 are retracted proximally and/or the second elongate element 120′ is advanced distally (e.g., by being pushed by the pusher 190) out of the distal end 181 of the deployment needle 180 to be positioned distal to the distal of the non-adherent anatomical structures to be brought into apposition. In some embodiments, the distal end 191 of the pusher 190 is advanced distally out of the distal end 181 of the deployment needle 180. The pusher 190 may be advanced distally to move the second elongate element 120′ with respect to the first elongate element 110′ to move the second elongate element 120 into a configuration transverse to the first elongate element 110′. In some instances, the pusher 190 may distally push the third element 130′ to cause the third element 130′ to move (e.g., pivot) the second elongate element 120′ with respect to the first elongate element 110′. Additionally or alternatively, the first elongate element 110′ may be retracted proximally to cause the second elongate element 120′ (e.g., positioned generally parallel to the first elongate element 110′) to ride along the third element 130′ and/or to move with respect to the distal surface of the distal of the non-adherent anatomical structures DS, PS and into a deployment position generally transverse to the first elongate element 110′. Once the second elongate element 120′ has been deployed, the deployment needle 180 (with the washer 130′ and the pusher 190 therein) may be proximally retracted through the puncture site through the non-adherent anatomical structures DS, PS to deploy the second elongate element 120′. The deployment needle 180 may be further proximally withdrawn relative to the pusher 190 and the apposition device 100′ once the distal end 181 of the deployment needle 180 is proximal to the proximal of the non-adherent anatomical structures DS, PS to deploy the third element 130′. The pusher 190 may then be distally advanced to move the third element 130′ distally with respect to the non-adherent anatomical structures DS, PS and the first elongate element 110′ to secure the non-adherent anatomical structures DS, PS in apposition with respect to each other, such as illustrated in FIG. 7. The first elongate element 110′ may be cut (e.g., in the case of a suture) or broken (e.g., in the case of a stylet or other frangible element) to leave a shortened portion thereof in place at the deployment site. It will be appreciated that any or all of the aspects of the above-described delivery and deployment may be performed using available appropriate imaging techniques such as known to those of ordinary skill in the art.

An apposition device 100, 100′ formed in accordance with various principles of the present disclosure can be left in place indefinitely or for a finite duration. In some embodiments, one or more components of the apposition device 100, 100′ is formed of a biodegradable material. For instance, the first elongate element 110, 100′ may be a biodegradable suture which naturally dissolves after a selected amount of time within the patient's body. The second elongate element 120, 120′ and third element 130 (if not a part of the biodegradable first elongate element 110, 100′) may be biodegradable as well, or may simply pass naturally out of the patient's body.

In some applications, a further device, such as a stent, is positioned across the non-adherent anatomical structures, such as to secure the structures together and/or to form an anastomosis therethrough or otherwise to allow passage of materials between the apposed anatomical structures. The further device may have retention members configured to hold the device in place with respect to the apposed anatomical structures, such as known to those of ordinary skill in the art. The retention members may be accessed and actuated (e.g., compressed) to allow removal of the further device as desired or medically indicated. In some instances, the further device may be left in place indefinitely or for a finite amount of time, such as one week, one month, six months, etc. One example of a further device is a stent which may be deployed after deployment of an apposition device 100, 100′ formed in accordance with various principles of the present disclosure. For instance, a stent may be used to form an anastomosis, such as gastrojejunostomy, across the anatomical structures (stomach S and jejunum J) of the example of an environment illustrated in FIG. 1. A pyloric occlusion device may be placed in the pylorus to inhibit or prevent passage of gastric materials into the duodenum and to redirect gastric matters to the jejunum in a manner known to those of ordinary skill in the art. The use of an apposition device as described above facilitates formation of such anastomosis by holding the jejunum in place with respect to the stomach as the stent is delivered from the stomach into the jejunum to form the anastomosis. In some instances, a natural anastomosis is formed across the apposed structures and/or the structures remain adhered to each other even after removal of the apposition device and/or the further device. It will be appreciated that other procedures (not necessarily forming an anastomosis) may also benefit from use of an apposition device 100 as described above.

Although embodiments of the present disclosure may be described with specific reference to medical devices and systems and procedures for treating the gastrointestinal system, it should be appreciated that such medical devices and methods may be used to treat tissues of the abdominal cavity, digestive system, urinary tract, reproductive tract, respiratory system, cardiovascular system, circulatory system, and the like. For instance, although the examples of embodiments illustrated and described herein are deployed to hold a stomach S and a jejunum J in apposition, apposition devices 100, 100′ formed in accordance with various principles of the present disclosure may be used to hold other anatomical structures in apposition, such as, without limitation, a gall bladder and a portion of the small intestines or the stomach of a patient.

It is to be understood by one of ordinary skill in the art that the present discussion is a description of illustrative examples of embodiments only, and is not intended as limiting the broader aspects of the present disclosure. All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure.

As used herein, the singular forms “a,” “an,” and “the” are intended to include the plural forms as well, unless the context clearly indicates otherwise. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. The terms “comprises” and/or “comprising,” or “includes” and/or “including” when used herein, specify the presence of stated features, regions, steps, elements and/or components, but do not preclude the presence or addition of one or more other features, regions, integers, steps, operations, elements, components and/or groups thereof.

In view of the above, it should be understood that the various embodiments illustrated in the figures have several separate and independent features, which each, at least alone, has unique benefits which are desirable for, yet not critical to, the presently disclosed apposition devices, systems, and methods. Therefore, the various separate features described herein need not all be present in order to achieve at least some of the desired characteristics and/or benefits described herein. Only one of the various features may be present in a device or system formed in accordance with various principles of the present disclosure. Alternatively, one or more of the features described with reference to one embodiment can be combined with one or more of the features of any of the other embodiments provided herein. That is, any of the features described herein can be mixed and matched to create hybrid designs, and such hybrid designs are within the scope of the present disclosure. Moreover, throughout the present disclosure, reference numbers are used to indicate a generic element or feature of the disclosed embodiment. The same reference number may be used to indicate elements or features that are not identical in form, shape, structure, etc., yet which provide similar functions or benefits. Additional reference characters (such as letters, as opposed to numbers) may be used to differentiate similar elements or features from one another.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit, and scope of the disclosure. All such similar substitutes and modifications apparent to those skilled in the art are deemed to be within the spirit, scope, and concept of the disclosure as defined by the appended claims. Various further benefits of the various aspects, features, components, and structures of apposition devices, systems, and methods such as described above, in addition to those discussed above, may be appreciated by those of ordinary skill in the art.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A system for moving and holding a proximal anatomical structure and a distal anatomical structure in apposition with respect to each other, said system comprising:
an apposition device comprising a first elongate element and a second elongate element movably coupled with respect to each other;
a needle with a lumen therethrough in which at least said second elongate element is positionable; and
a pusher within the needle lumen positioned proximal to said second elongate element;
wherein at least one component of said system is structured and configured to facilitate movement of said second elongate element with respect to said first elongate element from a delivery position generally along said first elongate element to a deployed configuration transverse to said first elongate element.

2. The system of claim 1, wherein a side port is defined in a wall of said needle configured for deployment of said second elongate element therethrough.

3. The system of claim 2, wherein a ramp is provided within the needle lumen along said side port to facilitate deployment of said second elongate element from the needle lumen and pivoting of said second elongate member into the deployed configuration.

4. The system of claim 3, wherein said ramp is formed from a portion of the needle wall pushed inwardly into the needle lumen to form the port.

5. The system of claim 2, wherein the side port is defined proximally to a distal end of said needle a distance allowing deployment of said second elongate element within a small space distal to the distal anatomical structure.

6. The system of claim 1, wherein said apposition device further comprises a third element positioned proximal to said second elongate element and distal to said pusher and configured to push said second elongate element into the deployed position when pushed distally by said pusher.

7. The system of claim 1, further comprising a third element configured and positioned to hold the anatomical structures in apposition.

8. The system of claim 7, wherein said third element is an expanded element associated with said first elongate element and positioned proximal to the proximal anatomical structure to hold the anatomical structures in apposition.

9. The system of claim 8, wherein said first elongate element and said third element are configured to be fixed with respect to each other to hold the structures in apposition between said third element and said third element.

10. The system of claim 9, wherein said first elongate element and said third element include interengaging ratchet features.

11. The system of claim 1, wherein said needle is a fine gauge needle.

12. A system for moving and holding a proximal anatomical structure and a distal anatomical structure in apposition with respect to each other, said system comprising:
an apposition device comprising a first elongate element and a second elongate element movably coupled with respect to each other; and
a needle with a lumen therethrough in which at least said second elongate element is positionable;
wherein a side port is defined in a wall of said needle configured for deployment of said second elongate element therethrough.

13. The system of claim 12, wherein a ramp is provided within the needle lumen along said side port to facilitate deployment of said second elongate element from the needle lumen and pivoting of said second elongate member into the deployed configuration.

14. The system of claim 13, wherein said ramp is formed from a portion of the needle wall pushed inwardly into the needle lumen to form the port.

15. The system of claim 12, further comprising a third element configured and positioned to hold the anatomical structures in apposition, said third element comprising an expanded element fixable with respect to said first elongate element proximal to the proximal anatomical structure to hold the anatomical structures in apposition.

16. A method of holding a first anatomical structure and a second anatomical structure in apposition with respect to each other with an apposition device comprising a first elongate element and a second elongate element movably coupled with each other, said method comprising:
delivering the second elongate element of the apposition device within a lumen of a needle to a deployment position distal to the second anatomical structure;
extending the second elongate element out a side port formed in a wall of the needle;
causing the second elongate member to move to a position transverse to the first elongate element;
proximally withdrawing the needle proximal to the anatomical structures; and
pulling the first elongate element proximally to draw the second elongate element proximally to move the second anatomical structure into apposition with the first anatomical structure.

17. The method of claim 16, wherein the first elongate element extends proximally along the outside of the needle, said method further comprising pulling the first elongate element proximally to cause the second elongate member to pull the second anatomical structure in apposition with the first anatomical structure.

18. The method of claim 17, further comprising positioning a third element with respect to the first elongate element and the first anatomical structure to hold the anatomical structures in apposition.

19. The method of claim 16, further comprising advancing a pusher distally through the needle lumen to push the second elongate element out of the side port.

20. The method of claim 19, further comprising pushing the second elongate element along a ramp within the needle lumen to advance the second elongate element out the side port defined in the needle wall.
